(19) United States
(12) Patent Application Publication
(10) Pub. No.: US 2024/0164935 A1
(43) Pub. Date: May 23, 2024

(54) FLUID COLLECTION DEVICES HAVING A SUMP BETWEEN A TUBE OPENING AND A BARRIER, AND RELATED SYSTEMS AND METHODS

(71) Applicant: PUREWICK CORPORATION, Covington, GA (US)

(72) Inventor: Camille Rose Newton, Bonsall, CA (US)

(21) Appl. No.: 18/426,795

(22) Filed: Jan. 30, 2024

Related U.S. Application Data
(63) Continuation of application No. 18/299,788, filed on Apr. 13, 2023, now Pat. No. 11,925,575.
Application No. 18/299,788 is a continuation of application No. PCT/US2021/043893, filed on Jul. 30, 2021.
(60) Provisional application No. 63/154,248, filed on Feb. 26, 2021.

(51) Int. Cl. A61F 5/453; A61F 5/44; A61F 5/443; A61F 5/451

(52) U.S. Cl. CPC A61F 5/453; A61F 5/4404; A61F 5/4401; A61F 5/443; A61F 5/451

(57) ABSTRACT

Examples relate to fluid collection devices, and related systems and methods. A fluid collection device includes a fluid impermeable barrier, a fluid permeable body, a tube, and a sump. The fluid impermeable barrier has a distal end region and defines an opening and a chamber in fluid communication with the opening. The fluid permeable body is positioned within the chamber at least partially at the distal end region. The tube extends into the chamber and has an end positioned proximate to the distal end region of the fluid impermeable barrier. The tube includes a tube opening at least proximate to the end of the tube and oriented to face at least a portion of the fluid permeable body. The sump is positioned in the area between the tube opening and the distal end region.

### CROSS-REFERENCE TO RELATED APPLICATIONS

This application is a continuation of U.S. application Ser. No. 18/299,788 filed on Apr. 13, 2023, which is a continuation of PCT International Application No. PCT/US2021/043893 filed on Jul. 30, 2021, which claims priority to U.S. Provisional Patent Application No. 63/154,248 filed on Feb. 26, 2021, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection devices continue to seek new and improved devices, systems, and methods to collect urine.

### SUMMARY

Embodiments disclosed herein are related to fluid collection devices having a defined sump, and related systems and methods. In an embodiment, a fluid collection device includes a fluid impermeable barrier, a fluid permeable body, a tube, and a sump. The fluid impermeable barrier at least partially defines an opening and a chamber within the fluid collection device in fluid communication with the opening, and the fluid impermeable barrier having a proximal end region and a distal end region. The fluid permeable body is positioned within the chamber and extends at least partially between the distal end region and the proximal end region. The tube extends into the chamber and has an end positioned proximate to the distal end region of the fluid impermeable barrier. The tube includes a tube opening proximate to the end of the tube and oriented to face at least a portion of the fluid permeable body. The sump is positioned in the area between the tube opening and the distal end region of the fluid impermeable barrier.

In an embodiment, a method of collecting fluid includes positioning a fluid collection device on skin of a user. The fluid collection device includes a fluid impermeable barrier at least partially having a proximal and a distal end region and defining an opening and a chamber within the fluid collection device in fluid communication with the chamber. The fluid collection device also includes a fluid permeable body positioned within the chamber and extending at least partially between the distal end region and the proximal end region. The fluid collection device also includes a tube extending into the chamber and having an end positioned proximate to the distal end region of the chamber. The tube includes a tube opening at least proximate to the end of the tube and oriented to face at least a portion of the fluid permeable body. The method includes collecting fluid discharged from the user in the chamber of the fluid collection device and drawing the fluid from the fluid collection device through the tube.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a block diagram of a fluid collection system, according to an embodiment.

FIG. 2A is a front isometric view of a male urine collection device, according to an embodiment.

FIG. 2B is a cross-sectional view of distal end region of a male fluid collection device, according to an embodiment.

FIG. 3A is a cross-sectional view of a distal end region of a fluid collection device, according to an embodiment.

FIG. 3B is a cross-sectional view of a distal end region of a fluid collection device, according to an embodiment.

FIG. 3C is a cross-sectional view of a distal end region of a fluid collection device, according to an embodiment.

FIG. 3D is a cross-sectional view of a distal end region of a fluid collection device, according to an embodiment.

FIG. 4 is a front isometric view of a distal end region of a fluid permeable body, according to an embodiment.

FIG. 5 is a front isometric view of a distal end region of a fluid collection device, according to an embodiment.

FIG. 6 is a cross-sectional view of a distal end region of a fluid collection device, according to an embodiment.

FIG. 7 is a schematic cross-sectional view of a portion of an embodiment of a tube, according to an embodiment.

FIG. 8A is a side view of a female fluid collection assembly, according to an embodiment.

FIG. 8B is a cross-sectional schematic of a distal end region of the fluid collection assembly of FIG. 8A.

FIG. 9 is a flow diagram of a method for collecting fluid with a fluid collection device, according to an embodiment.

### DETAILED DESCRIPTION

Embodiments disclosed herein are related to fluid collection devices having a defined sump, and related systems and methods. A fluid collection device may include a fluid impermeable barrier, a fluid permeable body, and a tube. The fluid impermeable barrier at least partially defines an opening and a chamber within the fluid collection device in fluid communication with the chamber, and includes a proximal end region and a distal end region. A fluid permeable body is positioned within the chamber and extends at least partially between the distal end region and the proximal end region. The tube extends into the chamber and has an end positioned in the chamber proximate to the distal end region of the fluid impermeable barrier. The tube also includes a tube opening (e.g., inlet) proximate to the end of the tube and oriented to face at least a portion of the fluid permeable body.

A defined sump of the fluid collection device may include an area or region within the urine collection device where fluid may collect for removal from the fluid collection device. A fluid collection device may include, from top to bottom in use, a top portion of the fluid impermeable barrier, a portion of the fluid permeable body uncovered or spaced from the top portion to allow discharged urine to enter to the fluid permeable body, a tube opening on a vacuum tube, and at least a portion of the fluid permeable body positioned between the tube opening and a bottom layer of the fluid impermeable barrier of the fluid collection device. For example, fluid may be received on or in the fluid permeable body, and be drawn to the defined sump as fluid is drawn from the sump with the tube. In some embodiments, the defined sump may include at least an area or region within the chamber having the tube opening of the tube spaced from the distal end region of the fluid impermeable barrier. Thus, when fluid collects within the sump between the tube opening and the distal end region of the fluid impermeable barrier, the tube may remove the fluid from the sump. In some embodiments, the fluid permeable body extends between the tube opening and the distal end region of the fluid impermeable barrier, resulting in the technical effect of the fluid permeable body providing connections for molecular bonding of water in the fluid. This configuration of the fluid permeable body between the tube opening and the fluid impermeable barrier also results in the fluid being drawn into the tube through the tube opening drawing fluid in other regions of the fluid permeable body to the sump via the molecular bonding of the water in the fluid. A defined sump within embodiments of fluid collection devices described herein also may provide the technical effect of preventing a loss of vacuum on fluid being drawn from the fluid collection device, thereby improving fluid capture from the fluid collection device. A defined sump within embodiments of fluid collection devices described herein also provides for a fluid permeable body that results in the technical effect of a network promoting connection of molecular bonding of the fluid being collected, thereby improving fluid removal from the fluid collection device.

In conventional fluid collection devices, fluid is often difficult or inefficient to remove from the fluid collection device when the fluid collection device is in a substantially horizontal position. In some embodiments of fluid collection devices described herein, the fluid collection devices may include a horizontal sump for a vacuum assisted fluid collection device that results in the technical of effect of efficient operation of the fluid collection devices when in a substantially horizontal position. For example, some embodiments of the fluid collection device may be configured to for use as a skin or wound care fluid collection device that may be placed on skin such as the abdomen, interior thigh, or the chest, where there is typically no lowest point for liquid to pool in the fluid collection device. In some embodiments, the fluid collection device may include a male external catheter that may be generally horizontal during use, such as positioned on the scrotal area or on either side of the penis or above the penis (below the umbilicus). A defined horizontal sump may maximize fluid capture by the fluid collection device by optimizing the location and angle of contact of the tube opening within the sump.

A fluid collection device used in a horizontal position and having a horizontal sump may include, in order from either top to bottom or bottom to top, a portion of the fluid permeable body uncovered to allow fluid to enter to the fluid permeable body, a tube opening on a vacuum tube, and at least a portion of the fluid permeable body positioned between the tube opening and a fluid impermeable barrier of the fluid collection device. In use, the horizontal sump may not be the lowest point of the fluid collection device, but rather the region in the horizontal sump where fluid is collected. For example, when a vacuum is applied to the fluid permeable body, the entire fluid permeable body between the tube opening and the fluid impermeable barrier may be the horizontal sump. For example, in a male external catheter urine collection device, a top side of the fluid permeable body may be generally uncovered (or spaced from a top portion of the fluid impermeable barrier) to receive discharged urine, and the bottom side of the fluid permeable body may be positioned against the bottom portion of the fluid impermeable barrier. The horizontal sump, then, may include the entire region of the fluid permeable body between the opening in the fluid impermeable barrier and the distal end region of the fluid impermeable barrier.

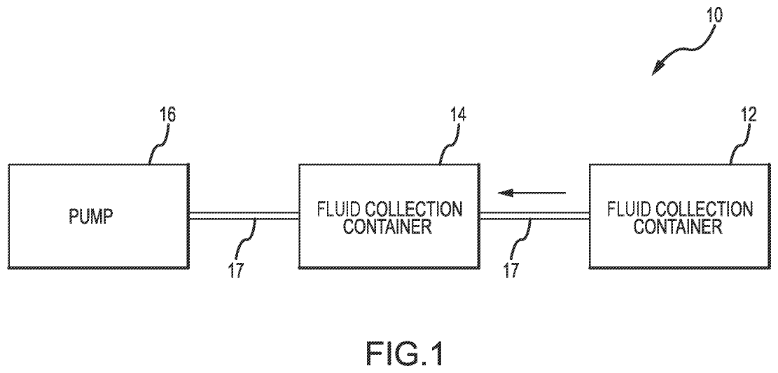

FIG. 1 is a block diagram of a fluid collection system 10, according to an embodiment. The fluid collection system 10 may be included in any of the embodiments of fluid collection systems described herein. The system 10 includes a fluid (e.g., urine) collection device 12 (e.g., any of the fluid collection assemblies disclosed herein), a fluid collection container 14, and a pump 16 (or vacuum). The fluid collection device 12, the fluid collection container 14, and the pump 16 may be fluidly coupled to each other via one or more tubes 17 or conduits. For example, fluid collection device 12 may be operably coupled to one or more of the fluid collection container 14 or the pump 16 via the tube 17. In some embodiments, the pump 16 may be secured directly to the fluid collection container 14. Fluid (e.g., urine or other bodily fluids) collected in the fluid collection device 12 may be removed from the fluid collection device 12 via the tube 17 secured to the fluid collection device 12. Suction force may be introduced into the chamber of the fluid collection device 12 via the inlet of the tube 17 responsive to suction (e.g., vacuum) force applied at the outlet of the tube 17.

The suction force may be applied to the outlet of the tube 17 by the pump 16 either directly or indirectly. The suction force may be applied indirectly via the fluid collection container 14. For example, the outlet of the tube 17 may be disposed within or fluidly coupled to an interior region of the urine collection container 14 and an additional tube 17 may extend from the urine collection container 14 to the pump 16. Accordingly, the pump 16 may apply suction to the fluid collection device 12 via the fluid collection container 14. The suction force may be applied directly via the pump 16. For example, the outlet of the tube 17 may be disposed within the pump 16. An additional tube 17 may extend from the pump 16 to a point outside of the fluid collection device 12, such as to the fluid collection container 14. In such examples, the pump 16 may be disposed between the fluid collection device 12 and the fluid collection container 14.

The fluid collection container 14 is sized and shaped to retain a fluid therein. The fluid collection container 14 may include a bag (e.g., drainage bag), a bottle or cup (e.g., collection jar), or any other enclosed container for storing bodily fluid(s) such as urine. In some examples, the tube 17 may extend from the fluid collection device 12 and attach to the fluid collection container 14 at a first point therein. An additional tube 17 may attach to the urine collection container 14 at a second point thereon and may extend and attach to the pump 16. Accordingly, a vacuum (e.g., suction) may be drawn through fluid collection device 12 via the fluid collection container 14. Fluid, such as urine, may be drained from the fluid collection device 12 using the pump 16.

The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more of a power cord (e.g., connected to a power socket), one or more batteries, or even manual power (e.g., a hand operated vacuum pump). In some examples, the pump 16 may be sized and shaped to fit outside of, on, or within the fluid collection device 12. For example, the pump 16 may include one or more miniaturized pumps or one or more micro pumps. The vacuum sources disclosed herein may include one or more of a switch, a button, a plug, a remote, or any other device suitable to activate the pump 16.

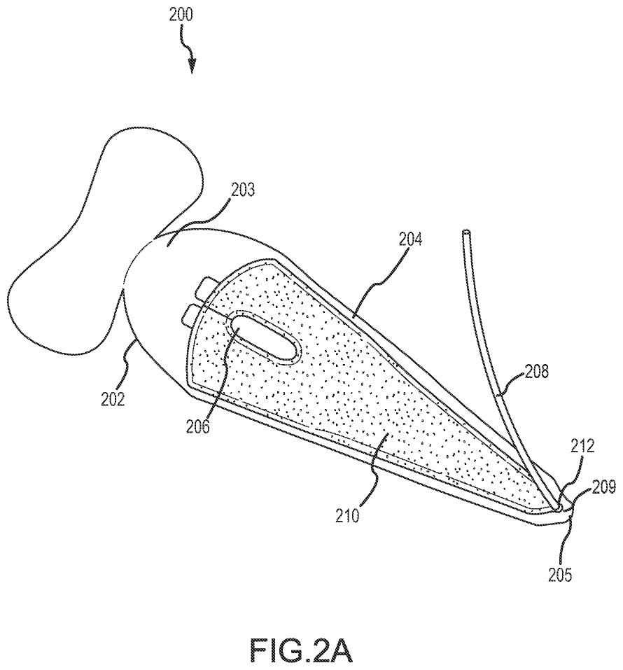

FIG. 2A is a front view of a male urine collection device 200, according to an embodiment. The urine collection device 200 includes a fluid impermeable barrier 202 at least partially defining an opening 206 and a chamber 204 within the fluid collection device in fluid communication with the opening 206. In some embodiments, a bottom portion of the fluid impermeable barrier 202 may define the opening 206 and an opposite or a top portion of the fluid impermeable bather 202 may define an aperture 212. The fluid impermeable barrier 202 includes a proximal end region 203 and a distal end region 205. The opening 206 may be positioned proximate or closer to the proximal end region 203 than the distal end region 205 of the fluid impermeable barrier 202, and the aperture 212 may be positioned proximate or closer to the distal end region 205 than the proximal end region 203. In some embodiments, the fluid impermeable barrier 202 narrows between the proximal end region 203 and the distal end region 205. For example, the fluid impermeable barrier 202 (and the chamber 204) may include a substantially triangular front profile, with the distal end region 205 being at the narrow end or tip of the triangular profile. The fluid impermeable bather 202 may include a shape substantially complementary to the chamber 204, such as a substantially triangular front profile. The fluid impermeable barrier 202 may include a substantially flexible fluid impermeable material, such as a fluid impermeable polymer (e.g., silicone, polypropylene, polyethylene, polyethylene terephthalate, a polycarbonate, etc.), polyurethane films, thermoplastic elastomer, oil, another suitable material, or combinations thereof. In some embodiments, the fluid impermeable barrier includes a paper-like or bag-like fluid impermeable material and/or a fluid impermeable fabric.

The urine collection device 200 also includes a fluid permeable body 210 positioned within the chamber 204 and extending at least partially between the distal end region 205 and the proximal end region 203. The fluid permeable body 210 may be shaped generally complementary to the shape of the chamber of the fluid impermeable barrier 202. In some embodiments, the fluid permeable body 210 is spaced from the edges of the fluid impermeable barrier 202 that extend at least partially between the distal end region 205 and the proximal end region 203. In some embodiments, the fluid permeable body 210 is positioned to abut the edges of the fluid impermeable barrier 202 such that fluid impermeable barrier 202 retains fluid in the fluid permeable body 210 from the opening 206 to the sump 209.

The fluid permeable body 210 can be configured to wick and/or allow transport of fluid away from the opening 206, thereby preventing the fluid from escaping the chamber 204. The fluid permeable body 210 also can wick and/or allow transport of the fluid generally towards the sump 209. The fluid permeable body 210 can include any material that can wick and/or allow transport of the fluid. The permeable properties referred to herein can be wicking, capillary action, diffusion, or other similar properties or processes, and are referred to herein as “permeable” and/or “wicking.” Such “wicking” or other physical properties may exclude absorption into the fluid permeable body 210, such as not include adsorption of the bodily fluids into the fluid permeable body 210. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 210 (e.g., absorbency), such as less than about 30 wt % of the dry weight of the wicking material, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 210. In an embodiment, the fluid permeable body 210 may include at least one absorbent or adsorbent material.

The fluid permeable body 210 can include a one-way fluid movement fabric. As such, the fluid permeable body 210 can remove fluid from the area around the penis, thereby leaving the area and urethra dry. The fluid permeable body 210 can enable the fluid to flow generally towards the sump 209 and the tube 208 within the chamber 204. The fluid permeable body 210 can include a porous or fibrous material, such as hydrophilic polyolefin. In some embodiments, the fluid permeable body 210 consists of or consists essentially of a porous or fibrous material, such as hydrophilic polyolefin. Examples of polyolefin that can be used in the fluid permeable body 210 include, but are not limited to, polyethylene, polypropylene, polyisobutylene, ethylene propylene rubber, ethylene propylene diene monomer, or combinations thereof. Moreover, the fluid permeable body 210 can be manufactured according to various manufacturing methods, such as molding, extrusion, or sintering. The fluid permeable body 210 can include varying densities or dimensions.

In some embodiments, the fluid permeable body 210 can include two or more layers of fluid permeable materials. For example, the fluid permeable body 210 can include a fluid permeable membrane covering or wrapped around a fluid permeable support, with both the fluid permeable membrane and the fluid permeable support being disposed in the chamber 204. The fluid permeable membrane can cover or extend across at least a portion (e.g., all) of at least the side of the fluid permeable support facing the penis of the user. The fluid permeable membrane and the fluid permeable support can be configured to wick any fluid away from the opening 206, thereby preventing the fluid from escaping the chamber 204 and promoting removal of the fluid through the tube 208. The permeable properties referred to herein can be wicking, capillary action, diffusion, or other similar properties or processes, and are referred to herein as “permeable” and/or “wicking.”

The fluid permeable membrane and the fluid permeable support also can wick the fluid generally towards an interior of the chamber 204, such as the sump 209. The fluid permeable membrane can include any material that can wick the fluid. For example, the fluid permeable membrane can include fabric, such as a gauze (e.g., a silk, linen, polymer based materials such as polyester, or cotton gauze), nylon (such as a spun nylon fibers), another soft fabric (e.g., jersey knit fabric or the like), or another smooth fabric (e.g., rayon, satin, or the like). Forming the fluid permeable membrane from gauze, soft fabric, and/or smooth fabric can reduce chaffing caused by the urine collection device 200. Other embodiments of fluid permeable membranes and fluid permeable supports are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 210 includes a fluid permeable support including a porous spun nylon fiber structure and a fluid permeable wicking membrane including gauze at least partially enclosing the spun nylon fiber structure.

The urine collection device 200 also includes a tube 208 extending into the chamber 204 and having an end 218 positioned proximate to the distal end region 205 of the fluid impermeable barrier 202. As shown in FIG. 2B, the tube 208 includes a tube opening 219 proximate to the end 218 of the tube 208. Returning to FIG. 2A, a sump 209 may be defined as the region or area between the tube opening 219 and the distal end region 205 of the fluid impermeable barrier 202. At least some of the fluid permeable body 210 may extend into the sump 209. Fluid discharged on the fluid permeable body 210 may flow to and pool in the sump 209 for removal when a vacuum is applied on the tube 208. The tube 208 may extend through an aperture 212 in a top portion of the fluid impermeable barrier 202 and into the chamber 204. In some embodiments, a first portion of the tube 208 including the end 218 and the tube opening 219 may be fixedly connected to the fluid impermeable barrier proximate to the aperture 212 and a second portion of the tube 208 may removably secured or connected to the fluid collection device 200 proximate to the aperture 212. The aperture 212 may be positioned proximate to the distal end region 205 of fluid impermeable barrier 202. In some embodiments, the end 218 of the tube 208 and/or the tube opening 219 are positioned between the distal end region 205 and the aperture 212. In some embodiments, the aperture 212 is absent, and the tube 208 may exit the chamber 204 through the opening 206. U.S. Provisional Patent 63/067,542 describes embodiments of male external catheter urine collection devices, aspects of which may be used in any of the embodiments disclosed herein, the disclosure of which are incorporated in their entirety by this reference.

Turning to FIG. 2B, which is a side view of a distal end of the urine collection device 200. As urine is discharged onto the fluid permeable body 210, the urine may flow to the lowest point to form a pool 50 of urine at the distal end region 205 of the fluid impermeable barrier 202. The end 218 of the tube 208 and the tube opening 219 are spaced from the distal end region 205 of the fluid impermeable barrier 202 such that a sump 209 for the pool 50 of urine is defined between the tube opening 219 and/or the end 218 of the tube 208.

In some embodiments, at least a portion of the fluid permeable body 210 is positioned between the tube opening 219 (and/or the end 218 of the tube 208) and the distal end region 205 of the fluid impermeable barrier 202. The tube opening 219 may be oriented to face at least the portion of the fluid permeable body 210, which may be positioned between the tube opening 219 and the distal end region 205 of the fluid impermeable barrier 202. In some embodiments, the fluid permeable body 210 may cover the tube opening 219. As used herein, covering of the tube opening 219 by the fluid permeable body 210 includes extending at least partially (e.g., entirely) across the tube opening 219, even if the tube opening 219 is oriented downward. That is, the fluid permeable body 210 may over the tube opening 219 below the tube opening 219. The fluid permeable body 210 may include a recess 226 proximate to the distal end region 205 and the tube opening 219 may be positioned at least partially (e.g., entirely) within the recess 226 with a distal portion or tip 225 of the fluid permeable body 210 between the tube opening 219 and the distal end region 205 of the fluid impermeable bather 202. Accordingly, the tube opening 219 may be embedded within the fluid permeable body 210. In some embodiments, the fluid permeable body 210 includes a bottom portion 224 extending from the distal end region 205 towards the proximal end region 203. The bottom portion 224 of the fluid permeable body 210 may interface a bottom portion 201 of the fluid impermeable barrier 202, which rests or is positioned on a user. The bottom portion 224 of the fluid permeable body 210 may be substantially planar. The fluid permeable body 210 also may include a lip 222 positioned proximate to the distal end region 205 fluid impermeable barrier 202. The recess 226 may be at least partially defined by the fluid permeable body 210 between the lip 222 and the bottom portion 224 of the fluid permeable body 210.

In some embodiments, the distal portion 225 of the fluid permeable body 210 is shaped substantially complementary to the distal end region 205. The distal portion 225 of the fluid permeable body 210 may be spaced from the distal end region 205 of the fluid impermeable barrier 202, such that a portion of the sump 209 is devoid of the fluid permeable body 210. In some embodiments, the distal portion 225 of the fluid permeable body 210 interfaces at least a portion (e.g., all) of the fluid impermeable barrier 202 at the distal end region 205.

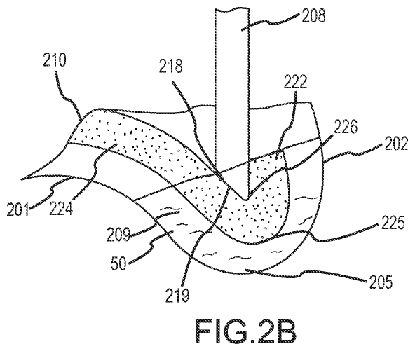

In some embodiments, the end 218 of the tube 208 includes a beveled end 218 with the tube opening 219 positioned at the beveled end 218. Whereas the end of a conventional tube is typically perpendicular to the sidewall of the conventional tube, the beveled end 218 of the tube 208 is at an acute or obtuse angle relative to the sidewall of the tube 208. With the beveled end 218 being angled relative to the sidewall of the tube 208, when the beveled end 218 and the tube opening 219 are generally horizontal, the tube 208 may angle relative to the bottom portion 224 of the fluid permeable body 210 and/or the bottom portion 201 of the fluid impermeable barrier 202. For example, the tube 208 may angle from the beveled end 218 towards the aperture 212 and out of the chamber 204, similar to the embodiment shown in FIG. 3A.

Embodiments having a beveled end 218 may be used when the urine collection device 200 is positioned generally horizontal on a user. In operation, when a vacuum is applied to the tube 208, the tube 208 draws fluid from the sump 209 into the tube 208 through the tube opening 219. The fluid permeable body 210 provides connections for molecular bonding of fluid (e.g., urine) in the chamber 204. The urine being drawn into the tube 208 through the tube opening 219, then, also may draw urine in the fluid permeable body 210 to the sump 209 via the molecular bonding of the water in the urine. The configuration of the sump 209 within the urine collection device 200 may prevent a loss of vacuum on fluid being drawn from the urine collection device 200, thereby improving fluid capture from the fluid collection device. In some embodiments, the distal end region 205 of the fluid impermeable barrier 202 may be the low point of the urine collection device 200 during use, and the tube 208 may include an end that is generally perpendicular to the sidewall of the tube 208.

FIG. 3A shows a distal end region 305 of a urine collection device 300 in a substantially horizontal position during use. Although only the distal end region 305 of the fluid collection device 300 is shown, unless otherwise noted, the fluid collection device 300 may include any aspect of the fluid collection device 200. For example, the fluid collection device 300 may include a fluid impermeable barrier 302 defining an opening (not shown) on a bottom portion 301 of the fluid impermeable barrier 302 and an aperture 312 on a top portion 303 of the fluid impermeable barrier 302 having a distal end region 305, a chamber 304, a fluid permeable body 310, and a tube 308 having an end 318 and a tube opening 319 that, unless otherwise noted, include any aspect of the fluid impermeable barrier 202 defining the opening 206 and the aperture 212, the chamber 204, the fluid permeable body 210, and the tube 208 having the end 218 and the opening 219 of the urine collection device 200. The fluid impermeable barrier 302 may include a bottom portion 301 and a top portion 303, with the chamber 304 defined between the bottom portion 301 and the top portion 303. The bottom portion 301 may be positioned on the user and may define the opening (not shown), and the top portion 303 may define the aperture 312 positioned proximate to the distal end region 305 of the fluid impermeable barrier 302.

The fluid permeable body 310 may include bottom portion 324 positioned on at least a portion of the bottom portion 301 of the fluid impermeable barrier 302. The fluid permeable body 310 may include a recess 326 proximate to the distal end region 305 of the fluid impermeable barrier 302 and the tube opening 319 may be positioned at least partially within the recess 326. The fluid permeable body 310 also may include a lip 322 positioned proximate to the distal end region 305 of the fluid impermeable barrier 302. The lip 322 may interface or be positioned against part of the top portion 303 of the fluid impermeable barrier 302. The recess 326 may be at least partially defined by the distal portion 325 of the fluid permeable body 310 between the lip 322 and the bottom portion 324 of the fluid permeable body 310. In some embodiments, the fluid permeable body 310 curls from the bottom portion 324 to the lip 322. The lip 322 and the distal portion 325 of the fluid permeable body 310 may be shaped complementary to and interface with the distal end region 305 of the fluid impermeable barrier 302.

At least a portion of the fluid permeable body 310 is positioned between the tube opening 319 and the fluid impermeable barrier 302. The tube opening 319 may be oriented to face at least the portion of the fluid permeable body 310 that may be positioned between the tube opening 319 and distal end region 305 of the fluid impermeable barrier 302. For example, the bottom portion 324 and/or the distal portion 325 of the fluid permeable body 310 may be positioned between tube opening 319 and the fluid impermeable barrier 302. In some embodiments, the urine collection device 300 may be used in a generally horizontal position with the bottom portion 324 of the fluid permeable body 310 and the bottom portion 301 of the fluid impermeable barrier 302 substantially horizontal, as shown in FIG. 3A. In the generally horizontal position, the beveled end 318 of the tube 308 may be substantially horizontal and at least partially positioned in the recess 326. The tube 308 may angle from the beveled end 318 to the aperture 312 at an acute angle relative to the bottom portion 324 of the fluid permeable body 310 and/or the bottom portion 301 of the fluid impermeable barrier 302. When in a generally horizontal position, the sump 309 of the urine collection device 300 may be positioned at least partially in the chamber 304 at the distal end region 305 of between the tube opening 319 and the bottom portion 301 of the fluid impermeable barrier 302, with the bottom portion 324 of the fluid permeable body 310 positioned therebetween. In some embodiments, the bottom portion 324 of the fluid permeable body 310 may interface or be adjacent to the tube opening 319. For example, part of the bottom portion 324 of the fluid permeable body may cover or extend across at least a portion (e.g., all) of the tube opening 319. The beveled end 318 of the tube 308 and the positioning of the sump 309 between the beveled end 309 and the fluid impermeable barrier 302 allows urine 50 to be drawn through the fluid permeable body 310 to the sump 309, and drawn into the tube 308 through the tube opening 319 when a vacuum is applied to the tube 308. The end 318 of the tube 308 may be positioned proximate to the lateral margin or distal end of the sump 309. In some embodiments, only the distal portion 325 of the fluid permeable body 310 is positioned in the chamber 304 between the end 318 of the tube 308 and the distal end region 305 of the fluid impermeable barrier 302.

In some embodiments, the urine collection device 300 may be configured for use with the distal end region 305 of the fluid impermeable barrier 302 at a low point of the urine collection device 300 during use. When the distal end region 305 is at the low point of the urine collection device 300 during use, a tube having an end generally perpendicular to the sidewalls of the tube may be used to draw urine 50 from the urine collection device 300. For example, the end of the tube may be inserted into the recess 326 with the distal portion 325 of the fluid permeable body 310 positioned in the chamber 304 between the end of the tube and the distal end region 305 of the fluid impermeable barrier 302. When the distal end region 305 is at the low point of the urine collection device 300 during use, the sump 309 may be positioned in the chamber 304 between the end of the tube and the distal end region 305 of the fluid impermeable barrier 302. In some embodiments of the fluid collection device in a horizontal position, the sump 309 includes a region of the fluid permeable body 310 between the opening in the fluid impermeable barrier and the tube opening 319. The sump 309 may further extend in the fluid permeable body 310 to the end of the lip 322. The fluid permeable body 310 may promote molecular bonding of the fluid held in the fluid permeable body 310, and the tube opening 319 may abut and/or be adjacent to the fluid permeable body 310 such that molecular bonds are not broken by a space of air between the tube opening 319 and the fluid 50.

FIG. 3B shows a distal end region 305 of a urine collection device 330 in a substantially horizontal position during use. Although only the distal end region 305 of the fluid collection device 330 is shown, unless otherwise noted, the fluid collection device 330 may include any aspect of the fluid collection devices 200, 300. For example, the fluid collection device 330 may include a fluid impermeable barrier 302 having a bottom portion 301 and a top portion 303. The fluid impermeable barrier 302 may include the distal end region 305 and define an opening (not shown) and a chamber 304.

The urine collection device 330 may include a fluid permeable body 340. The fluid permeable body 340 may include any aspect of the fluid permeable bodies 210, 310, such as the materials of the fluid permeable body 210. The fluid permeable body 340 may include bottom portion 344 positioned on at least a portion of the bottom portion 301 of the fluid impermeable barrier 302, and a top portion 342 positioned adjacent to or interfacing at least a portion of the top portion 303 of the fluid impermeable barrier 302. A distal portion 345 of the fluid permeable body may connect the top portion 342 and the bottom portion 344 of the fluid permeable body 340. The fluid permeable body 340 may include tubular channel 346 extending between the top portion 342 and the bottom portion 344 of the fluid permeable body 340. The tubular channel 326 may extend into the fluid permeable body 340 to the distal portion 345 of the fluid permeable body 340. The fluid permeable body 340 may be shaped complementary to and interface with the distal end region 305 of the fluid impermeable barrier 302.

The urine collection device 330 also may include a tube 338 having a tube opening 349 spaced from an end 348 of the tube 338. In some embodiments, the end 348 of the tube 338 is capped, closed, or covered such that fluid communication through the end 348 of the tube 338 is absent. Rather than being positioned at the end 348 of the tube 338, the tube opening 349 may be defined and/or positioned in a sidewall of the tube 338. With the tube opening 349 in the sidewall of the tube 348, the tube opening 349 may be oriented towards the bottom portion 301 of the fluid impermeable barrier 302 and/or the bottom portion 344 of the fluid permeable body 340.

At least a portion of the fluid permeable body 340 is positioned between the tube opening 349 and the fluid impermeable barrier 302. The tube opening 349 may be oriented to face at least the portion of the fluid permeable body 340 that may be positioned in the chamber 304 between the tube opening 349 and distal end region 305 of the fluid impermeable barrier 302. For example, the bottom portion 344 may be positioned between tube opening 349 and the fluid impermeable barrier 302. In some embodiments, the urine collection device 330 may be used in a generally horizontal position with the bottom portion 344 of the fluid permeable body 340 and the bottom portion 301 of the fluid impermeable barrier 302 substantially horizontal, as shown in FIG. 3B. In the generally horizontal position, the covered or capped end 348 of the tube 338 may abut or interface the distal portion 345 of the fluid permeable body 340 in the tubular channel 346. The tube opening 349 in the sidewall of the tube 338 may be oriented downwards to the bottom portion 344 of the fluid permeable body 340 and the bottom portion 301 of the fluid impermeable barrier 302. The tube 338 may be substantially horizontal within at least the distal region of the tubular channel 348 as the tubular channel 348 approaches the distal end region 305 of the fluid impermeable barrier 302. In some embodiments, the tube 338 exits the chamber 304 through the opening (next to the penis).

When in a generally horizontal position, the sump 339 of the urine collection device 330 may be positioned at least partially in the chamber 304 proximate to the distal end region 305 between the tube opening 349 and the bottom portion 301 of the fluid impermeable barrier 302, with the bottom portion 344 of the fluid permeable body 340 positioned therebetween. The sump 339 may continue through the distal portion 345 of the fluid permeable body 340 as the fluid permeable body 330 wraps around the end 348 of the tube 338. In some embodiments, the bottom portion 344 of the fluid permeable body 340 may interface or be adjacent to the tube opening 349. For example, part of the bottom portion 344 of the fluid permeable body 340 may cover or extend at least partially (e.g., entirely) across the tube opening 349. The orientation of the tube opening 349 towards the bottom portion 301 of the fluid impermeable barrier 302 allows urine 50 to be drawn through the fluid permeable body 310 to the sump 339, and drawn into the tube 338 through the tube opening 349 when a vacuum is applied to the tube 338.

FIG. 3C shows a distal end region 305 of a urine collection device 360 in a substantially horizontal position during use. The urine collection device 360 may include the fluid permeable body 310 of the urine collection device 300, and the fluid impermeable barrier 302 and the tube 338 of the urine collection device 330. The capped or covered end 348 of the tube 338 may be positioned in the recess 326 of the fluid permeable body 310. A sump 369 may be positioned between the tube opening 349 and the bottom portion 301 of the fluid impermeable barrier 302, similar to the sump 339. Urine may be withdrawn from the urine collection device 360 when the urine collection device 360 is in the horizontal position, similar to the withdrawal of urine from the urine collection device 330, described above.

FIG. 3D shows a distal end region 305 of a urine collection device 370 in a substantially horizontal position during use. Although only the distal end region 305 of the fluid collection device 370 is shown, unless otherwise noted, the fluid collection device 370 may include any aspect of the fluid collection devices 200, 300, 330, 360. For example, the fluid collection device 370 may include a fluid impermeable barrier 302 having a bottom portion 301 and a top portion 302. The fluid impermeable barrier 302 may include the distal end region 305 and define an opening (not shown) and a chamber 304.

The urine collection device 370 may include a fluid permeable body 380. The fluid permeable body 380 may include any aspect of the fluid permeable bodies 210, 310, such as the materials of the fluid permeable body 210. The fluid permeable body 380 may include a bottom portion 384 positioned on at least a portion of the bottom portion 301 of the fluid impermeable barrier 302. The fluid permeable body 310 may include a recess 386 proximate to the distal end region 305 of the fluid impermeable barrier 302. The fluid permeable body 380 also may include a lip 382 positioned proximate to the distal end region 305 of the fluid impermeable barrier 302. The lip 382 may interface or be positioned against part of the top portion 303 of the fluid impermeable barrier 302. The recess 326 may be at least partially defined by the distal portion 385, the top portion 382, and the bottom portion 384 of the fluid permeable body 380. In some embodiments, the bottom portion 384, the distal portion 385, and the top portion 382 are three separate pieces or elements positioned within the chamber 304.

The urine collection device 370 also may include the tube 338 having the tube opening 349 spaced from the end 348 of the tube 338. The tube opening 349 may be oriented towards the bottom portion 301 of the fluid impermeable barrier 302 and/or the bottom portion 344 of the fluid permeable body 340. The bottom portion 384 may be positioned between tube opening 349 and the fluid impermeable barrier 302. In some embodiments, the urine collection device 370 may be used in a generally horizontal position with the bottom portion 384 of the fluid permeable body 380 and the bottom portion 301 of the fluid impermeable barrier 302 substantially horizontal, as shown in FIG. 3D. In the generally horizontal position, the covered or capped end 348 of the tube 338 may abut or interface the distal portion 385 of the fluid permeable body in the recess 386. The tube opening 349 in the sidewall of the tube 338 may be oriented downwards to the bottom portion 384 of the fluid permeable body 380 and the bottom portion 301 of the fluid impermeable barrier 302. The tube 338 may be substantially horizontal and may exit the chamber 304 through the opening (next to the penis).

A sump 379 may be positioned between the tube opening 349 and the bottom portion 301 of the fluid impermeable barrier 302, similar to the sumps 339, 369. Urine may be withdrawn from the urine collection device 370 when the urine collection device 370 is in the horizontal position, similar to the withdrawal of urine from the urine collection devices 330, 360, described above.

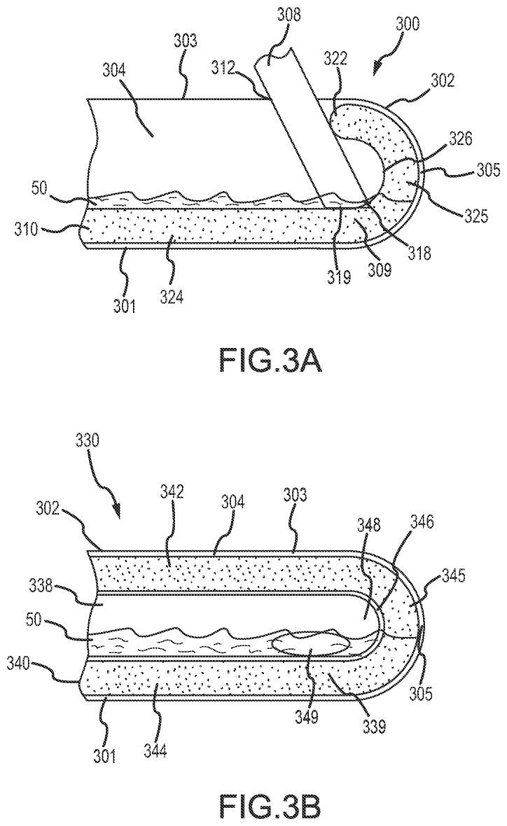

FIG. 4 shows a distal end of fluid permeable body 410 that may be used in a male urine collection device. For example, the fluid permeable body 410 may be positioned within the chambers 204, 304 of the fluid impermeable bathers 202, 302. The fluid permeable body 410 may include any of the materials described above in relation to the fluid permeable body 210. In some embodiments, the fluid permeable body 410 includes a first portion 422 and a second portion 424. The first portion 422 may be positioned proximate or adjacent to a bottom portion of the fluid impermeable barrier and the second portion 424 may be positioned proximate or adjacent to a top portion of the fluid impermeable barrier. Alternatively, the first portion 422 may be positioned proximate or adjacent to the top portion of the fluid impermeable barrier and the second portion 424 may be positioned proximate or adjacent to the bottom portion of the fluid impermeable barrier. An open region 414 may be positioned between the first portion 422 and the second portion 424 of the fluid permeable body 420. In some embodiments, the second portion 424 of the fluid permeable body 410 is absent, and the first portion 422 of the fluid permeable body 410 may be positioned adjacent the bottom portion of the fluid impermeable barrier with a channel wall 416 positioned proximate or adjacent to the top portion of the fluid impermeable barrier. In some embodiments, the fluid permeable body 410 may be used with the channel wall 416 proximate to the bottom portion of the fluid impermeable barrier.

The fluid permeable body 410 also may include a channel 412 sized to receive at least a portion of a tube 408. The channel 412 may be at least partially defined by the first portion 422 of the fluid permeable body 410. In some embodiments, the channel 412 may be defined by the top portion 422 of the fluid permeable body 410 and a channel wall 416. The channel 412 may be generally tubular. The channel wall 416 may include any of the materials of the fluid permeable body 210. In some embodiments, the channel wall 416 is integrally formed with the first portion 422 of the fluid permeable body 410 with the channel 412 between the first portion 422 and the channel wall 416. The channel 412 may extend from proximate to a distal end 425 of the fluid permeable body 410 towards the opening in the fluid impermeable barrier.

A urine collection device having the fluid permeable body 410 also may include a tube 408 configured to provide fluid communication between the sump 409 and chamber of the urine collection device and a vacuum source. The tube 408 may include any aspect of the tubes 208, 308 described herein. In some embodiments, the tube 408 includes an opening 419 at an end 418 of the tube 408 that is generally perpendicular to the sidewall of the tube 408 (e.g., a beveled end may be absent from the tube 408). When the fluid permeable body 410 and the tube 408 are positioned within a chamber of a fluid impermeable barrier, a sump 409 may be formed at least partially in the fluid permeable body 410 between the tube opening 419 and the a distal end of the fluid impermeable barrier. The channel 412 may form a pocket for the tube 408 that directs or biases the end 418 and the tube opening 419 to stay in contact with the fluid permeable body 410. The channel 412 also may end or terminate spaced or distanced from the distal portion 425 of the fluid permeable body 410 such that the tube opening 419 is kept spaced from the fluid impermeable barrier. In use, the fluid permeable body 410 may direct urine to the tube opening 419, similar to the fluid permeable body 210 described above in relation to the urine collection device 200.

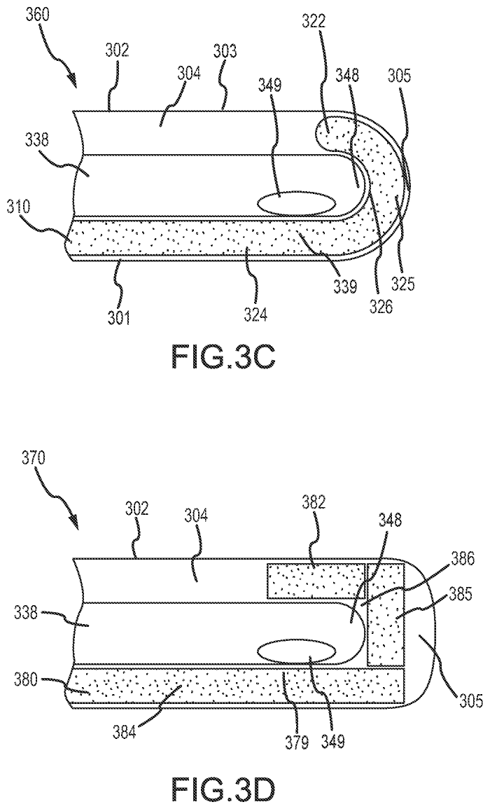

FIG. 5 is a front isometric view of the distal end of fluid collection device 530. Although only the distal end of the fluid collection device 530 is shown, unless otherwise noted, the fluid collection device 530 may include any aspect of the fluid collection device 200. In some embodiments, the fluid collection device 530 includes a fluid impermeable barrier 532 having a distal end region 535, a bottom portion 531, and a top portion 533. A chamber 534 and an aperture 539 may be at least partially defined by the fluid impermeable barrier 532.

The fluid collection device 530 also includes a fluid permeable body 540. Unless otherwise noted, the fluid permeable body 540 may include any aspect of the fluid permeable body 210, such as the materials and layers described in relation to the fluid permeable body 210. The fluid permeable body 540 may include a crescent-shaped protrusion 548 at least partially defining a pocket 546 between a portion of the crescent-shaped protrusion 548 and the distal portion 545 of the fluid permeable body 540. The pocket 546 may be sized to receive the end and at least a portion of the tube 508.

The tube 508 may extend through the aperture 539 in the top portion 533 of the fluid impermeable barrier 532 and into the pocket 546. The tube 508 may include flat end or a beveled end similar or the same as the beveled end of the tubes 208, 308. The end of the tube 508 may be positioned within the pocket 546 such that the tube opening at the end of the tube 508 is enveloped by the fluid permeable body 540 within the pocket 546. With the end of the tube 508 positioned in the pocket 546 and spaced from the distal end region 535 of the fluid impermeable barrier 532 and the bottom portion 531 of the fluid impermeable barrier 532, a sump 549 may be defined between the end of the tube 508 and the fluid impermeable barrier 532 in the distal end region 535 of the fluid impermeable barrier 532. Urine may be drawn into the tube 508 similar to other urine collection devices, such as the urine collection device 200.

In some embodiments of urine or fluid collection devices 200, 300, 330, 360, 370, 400, and 500, the fluid impermeable barrier defining of the chamber may include a silicone shell configured to retain its shape at the distal end region. In some embodiments of urine or fluid collection devices 200, 300, 330, 360, 370, 400, and 500, at least one of a wire coil or plastic may be positioned proximate the distal end of the fluid impermeable barrier and configured to inhibit collapse of the chamber proximate to the distal end region. In some embodiments of urine or fluid collection devices 200, 300, 330, 360, 370, 400, and 500, the chamber may include a filler material in a portion of the chamber proximate to the distal end region of the fluid impermeable barrier. The filler material may include at least one of netting, beads, or straws that inhibit collapse of the chamber proximate to the distal end region.

Aspects of fluid permeable bodies and the sumps of the urine or fluid collection devices 200, 300, 330, 360, 370, 400, and 500 may be applied to other urine collection devices. For example, PCT International Application No. PCT/US2019/029616, U.S. patent application Ser. No. 15/260,103, U.S. patent application Ser. No. 16/433,773, and U.S. Provisional Patent Application No. 63/067,542 describe various embodiments of both male and female fluid collection devices, including fluid permeable and fluid impermeable materials, that may be used in any of the embodiments disclosed herein, the disclosure of which are incorporated in their entirety by this reference. In some embodiments, the fluid collection device may include a female urine collection device having a substantially tubular fluid impermeable barrier defining an aperture at the proximal end of the fluid impermeable barrier and an opening positioned between the distal end region and the proximal end region of the fluid impermeable barrier. The fluid permeable body in the female urine collection device may include a substantially cylindrical portion extending across at least a portion (e.g. all) of the opening. The fluid permeable body also may include a distal portion defining a recess to receive the end of a tube therein, similar to the fluid permeable bodies 340, 350.

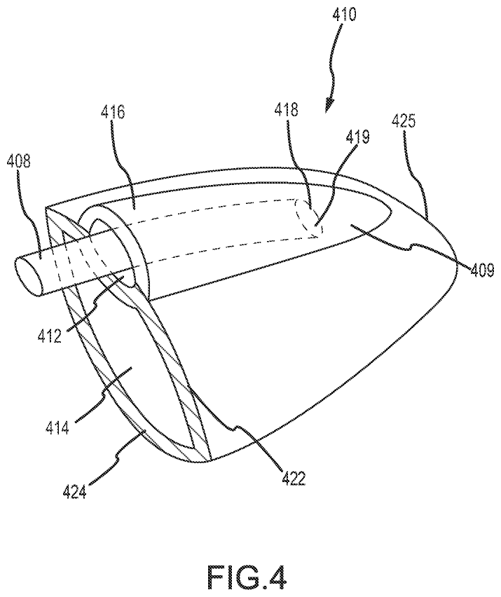

In some embodiments, a fluid collection device may be used to remove fluid from a wound or other regions of the body (besides the urethra) where fluids may collect or be discharged. FIG. 6 is a partial side view of a fluid collection device 600. The fluid collection device 600 may, for example, be used in removing wound drainage from a wound. The fluid collection device 600 may include a fluid impermeable barrier 602 defining an opening 606 and an aperture 612. The opening 606 may be positioned on a side of the fluid collection device 600 that is to be disposed on or at least proximate to the skin 60 of the user. The aperture 612 may be positioned substantially opposite to the opening 606. The fluid impermeable barrier 602 may include a distal end region 605 and may at least partially define a chamber 604 within the fluid collection device 600.

The fluid collection device 600 also includes a fluid permeable body 610. The fluid permeable body may include any aspect of the fluid permeable body 210, such as the materials of the fluid permeable body 210. The fluid permeable body 610 may include bottom portion 624 extending across at least a portion (e.g., all) of the opening 606 such that the bottom portion 624 of the fluid permeable body 610 may be positioned at least proximate to the skin 60. For example, a fluid permeable membrane of the fluid permeable body 610 may be disposed on the skin 60. The fluid permeable body 610 may include a recess 626 proximate to the distal end region 605 of the fluid impermeable barrier 602 and a tube opening 619 of a tube 608 may be positioned at least partially within the recess 626. The fluid permeable body 610 also may include a lip 622 positioned proximate to the distal end region 605 of the fluid impermeable barrier 602. The lip 622 may interface or be positioned against part of a top portion of the fluid impermeable barrier 602 and/or a portion of the fluid impermeable barrier 602 defining the distal end region 605 of the fluid impermeable barrier 602. The recess 626 may be at least partially defined by the distal portion 625 of the fluid permeable body 610 between the lip 622 and the bottom portion 624 of the fluid permeable body 610. In some embodiments, the fluid permeable body 610 curls from the bottom portion 624 to the lip 622. The lip 622 and the distal portion 625 of the fluid permeable body 610 may be shaped complementary to and interface with the distal end region 605 of the fluid impermeable barrier 602.

The tube 608 may extend through the aperture 612 into the chamber 604 and may include a beveled end 618. In some embodiments, a conduit including the end and the tube opening is fixedly formed in the chamber 604 extending from the top portion of the fluid impermeable barrier 602, and the tube 608 may be detachable secured to connected to the fluid collection device 600 proximate to the aperture 612. The tube opening 619 may be positioned at the beveled end 618 of the tube 608. The beveled end 618 of the tube 608 is at an acute or obtuse angle relative to the sidewall of the tube 608. With the beveled end 618 being angled relative to the sidewall of the tube 608, when the beveled end 618 and the tube opening 619 are generally horizontal and/or parallel with the bottom portion 624 of the fluid permeable body 610, the tube 608 may angle relative to the bottom portion 624 of the fluid permeable body 610. For example, the tube 608 may angle from the beveled end 618 towards the aperture 612 and out of the chamber 604.

Embodiments having the tube 608 with the beveled end 618 may be used when the fluid collection device 600 is positioned generally horizontal on a user. In operation, when a vacuum is applied to the tube 608, the tube 608 draws fluid from the sump 609 into the tube 608 through the tube opening 619. The fluid permeable body 610 provides connections for molecular bonding of fluids in the wound and/or in the chamber 604. The fluids being drawn into the tube 608 through the tube opening 619, then, also may draw fluid in the fluid permeable body 610 to the sump 609 via the molecular bonding of the water in the fluids. The configuration of the sump 609 within the urine collection device 600 may prevent a loss of vacuum on fluid being drawn from the fluid collection device 600 and/or the fluid permeable body 610 may promote molecular bonding of fluid in the sump 609 between the fluid permeable body 610 and the tube opening 619, thereby improving fluid capture from the fluid collection device 600.

In some embodiments, the tube 608 may be positioned generally horizontally within the chamber 604. For example, the tube 608 may include a tube opening spaced from an end of the tube 608, similar to or the same as the tube 338. The tube opening may be oriented towards the 606 and/or the bottom portion 624 of the fluid permeable body 610.

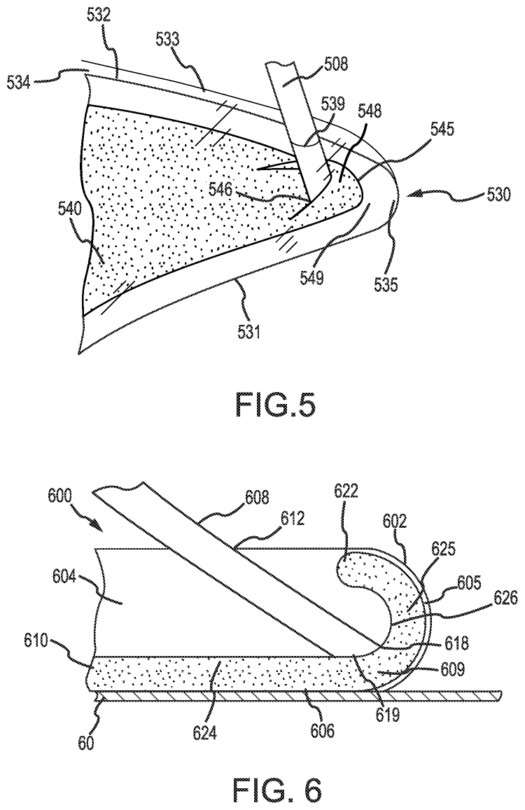

As noted above, some embodiments of a fluid collection device may include a tube having a tube opening offset from the end of the tube and/or on a sidewall of the tube (rather than defined at the terminating end of the tube). FIG. 7 is a schematic cross-sectional view of a portion of an embodiment of a tube 700, according to an embodiment. The tube 700 may be used in any of the fluid collection device described herein. Except as otherwise disclosed herein, the tube 700 may be the same or substantially similar to any of the tubes disclosed herein. For instance, the tube 700 may include at least one wall 702 defining an inlet 704 (or tube opening), an outlet (not shown), and a passageway 708 extending from the inlet 704 to the outlet. The tube 700 also includes at least one tube porous material 710 disposed in the passageway 708. It is noted that, while the inlet 704 of the tube 700 is illustrated and discussed, the same principles discussed herein with regards to FIG. 7 also applies to the outlet.

The wall 702 includes a laterally extending portion 702a and a terminal portion 702b. The laterally extending portion 702a extending generally parallel to a longitudinal axis 712 of the tube 700. The terminal portion 702b extends from the laterally extending portion 702a and forms a terminal end 720 of the tube 700. For example, the terminal portion 702b may extend from the laterally extending portion 702a in a direction that is obliquely angled or perpendicular to the longitudinal axis 712 of the tube 700.

The wall 702 may define the inlet 704. However, the inlet 704 is not at the terminal end 720 of the tube 700. Instead, the inlet 704 may be adjacent to (as shown) or spaced from the terminal end 720. The inlet 704 is defined by the laterally extending portion 702a of the wall 702 and, when the inlet 704 is adjacent to the terminal end 720, by the terminal portion 702b of the wall 702. It is noted that, in some embodiments, the tube porous material 710 may be omitted from the tube 700.

In some embodiments, the dimensions and/or cross-sectional shapes of some of the tubes disclosed herein may be different than the inlets and outlets of devices to which the tubes are configured to be attached. The different dimensions and/or cross-sectional shapes of the tubes relative to the inlets and outlets of the devices may prevent direct attachment of such tubes to the inlets and outlets of the devices. Further, the wall of some of the tubes disclosed here may exhibit a relatively small Young's modulus and/or a relatively small thickness which may inhibit forming an interference fit with the devices. As such, directly attaching such tubes to the devices may be difficult. Thus, the tubes disclosed herein may include one or more adaptors that are configured to facilitate attachment between the tubes disclosed herein and the inlets and/or outlets of the devices.

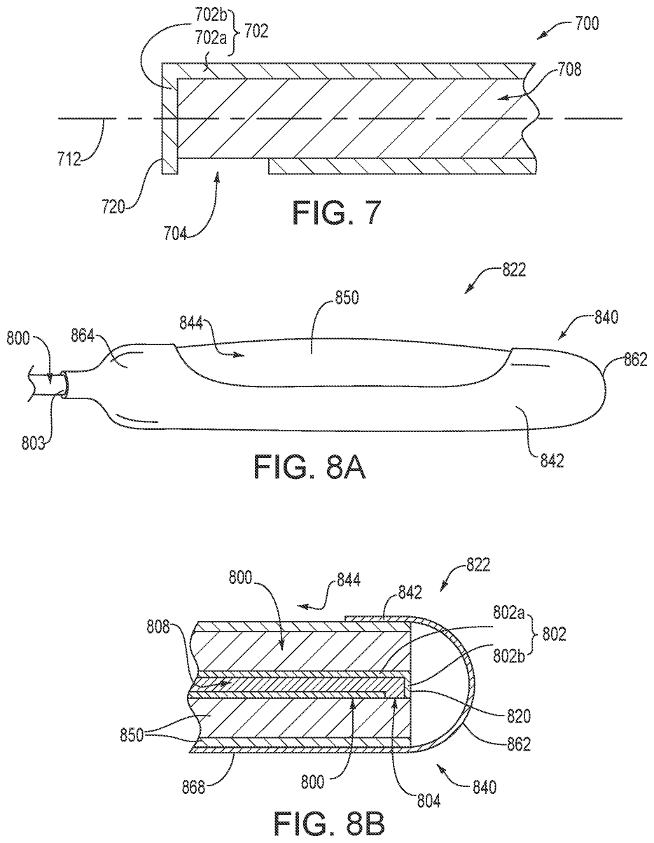

As noted above, aspects of the fluid collection devices described herein may be included in a female urine collection device, such as a female external catheter. FIG. 8A is a side view of a fluid collection system 822 in fluid communication with a fluid collection assembly 840. The fluid collection assembly 840 may include a female external catheter having a substantially tubular fluid impermeable barrier 842 having a proximal end region 864 and a distal end region 862. The fluid impermeable barrier 842 may define at least one opening 844, a chamber within the fluid collection assembly 840, and an aperture 803 at the proximal end region 864. The fluid collection assembly 840 also includes at least one fluid permeable body 850 disposed in the chamber.

FIG. 8B is a cross-sectional schematic of a distal end region 862 of the fluid collection system 822, according to an embodiment. The tube 800 may include at least one wall 802 defining an inlet 804 and a passageway 808 and a tube porous material 810 disposed in the passageway 808. Except as otherwise disclosed herein, the fluid collection assembly 840 is the same or substantially similar to any of the fluid collection assemblies disclosed herein. The tube 800 may be substantially similar to the tube 700 illustrated in FIG. 7. For example, the inlet 804 of the tube 800 is not at the terminal end 820 of the tube 800. Instead, the wall 802 includes a laterally extending portion 802a and a terminal portion 802b extending from the laterally extending portion 802a. The terminal portion 802b of the wall 802 defines the terminal end 820 of the tube 800. The laterally extending portion 802a at least partially defines the inlet 804 of the tube 800. The inlet 804 is adjacent to the assembly porous material 850 which prevents the need to offset the inlet 804 relative to a terminal end 866 of the assembly porous material 850. Further, over-insertion of the tube 800 into the fluid collection assembly 840 will result in the terminal portion 802b of the wall 802 contacting the fluid impermeable barrier 842 instead of the fluid impermeable barrier 842 obstructing the inlet 804.

In some embodiments, the fluid collection assembly 840 may be generally horizontally oriented. The fluid collection assembly 840 may be generally horizontally oriented when the distal end region 862 is not the gravimetric low point of the chamber 846. Instead, at least a portion of a back side 868 of the fluid impermeable barrier 842 (e.g., a side of the fluid impermeable barrier 842 generally opposite the opening 844) or an intersection between the back side 868 and the distal end region 862 is the gravimetric low point of the chamber 846. The fluid collection assembly 840 may be generally horizontal when the patient is sitting or standing. The bodily fluids may generally collect in a portion of the chamber 846 along the back side 868 of the fluid impermeable barrier 842 when the fluid collection assembly 840 is generally horizontally oriented. The inlet 804 of the tube 800 may be oriented to generally face the back side 868 of the fluid impermeable barrier 842. In some embodiments, the inlet 804 of the tube 800 may be oriented to generally face the front side of the fluid impermeable barrier 842 that at least partially defines the opening 844. The inlet 804 may remove bodily fluids from the chamber 846 sooner and more efficiently from the chamber 846 than conventional tubes when the fluid collection assembly 840 is generally horizontally oriented since the inlet 804 generally faces towards the pooling bodily fluids.

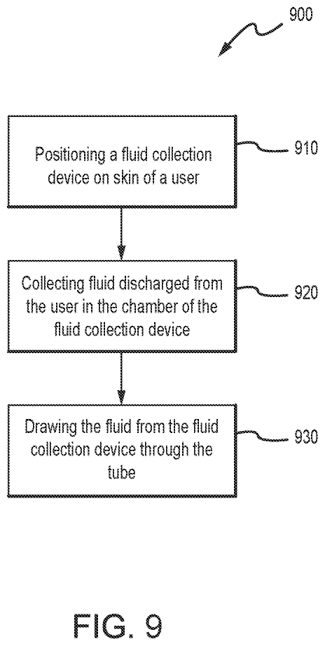

FIG. 9 is a flow diagram of a method 900 for collecting fluid from a user, according to an embodiment. The method 900 includes an act 910 of positioning a fluid collection device on skin of a user. The method 900 also includes an act 920 of collecting fluid discharged from the user in the chamber of the fluid collection device. The method also includes an act 930 of drawing the fluid from the fluid collection device through the tube.

Any of the fluid (including urine) collection devices described herein may be used in the method 900. In some embodiments, the fluid collection device of the method 900 includes a fluid impermeable barrier at least partially defining an opening and a chamber within the fluid collection device in fluid communication with the chamber, the fluid impermeable barrier having a proximal end region and a distal end region. The fluid collection device of the method 900 may include a fluid permeable body positioned within the chamber and extending at least partially between the distal end region and the proximal end region. The fluid collection device of the method 900 may include a tube extending into the chamber and having an end positioned proximate to the distal end. The tube may include a tube opening proximate to the end of the tube and oriented to face at least a portion of the fluid permeable body that may be positioned in the chamber between the distal end region of the fluid impermeable barrier and the end of the tube.

In some embodiments, the method 900 includes an act of covering the tube opening with the fluid permeable body and/or embedding the tube opening at least partially in the fluid permeable body. In some embodiments, the method 900 includes an act of inserting the tube at least partially through a channel in the fluid permeable body. In some embodiments, the method 900 includes inserting the tube opening in a recess in the fluid permeable body with the tube opening oriented to face a portion of the fluid permeable body that may be positioned between the tube opening and the distal end of the fluid impermeable barrier.

In some embodiments, the method 900 includes an act of inserting a penis of the user in the opening in the fluid impermeable barrier. The act 910 of positioning a fluid collection device on skin of a user may include positioning a bottom portion of the fluid impermeable barrier having a bottom portion to interface the user such that the bottom portion of the fluid impermeable barrier and a bottom portion of the fluid permeable body are substantially horizontal when the user is in a supine position. In some embodiments, the end of the tube may be capped or covered and the tube may include a sidewall defining the tube opening, with the tube opening being oriented towards the bottom portion of the fluid impermeable barrier and the tube being generally horizontal. In some embodiments, the fluid impermeable barrier defines an aperture positioned between the opening and the distal end, with the tube extending through the aperture into the chamber. In some embodiments, the end of the tube positioned in the chamber may include a beveled end with the tube opening positioned at the beveled end, and the tube may relative to the bottom portion of the fluid permeable body from the beveled end towards the aperture and out of the chamber.

In some embodiments, the act 910 of positioning a fluid collection device on skin of a user may include positioning the fluid collection device on a wound on the skin of the user with the opening in the fluid impermeable barrier over the wound and a bottom portion of the fluid permeable body disposed on the wound.

The acts of the method 900 described above are for illustrative purposes. For example, the acts of the method 900 can be performed in different orders, split into multiple acts, modified, supplemented, or combined. In an embodiment, one or more of the act of the method 900 can be omitted from the method 900. Any of the acts of the method 900 can include using any of the urine collection devices disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A male urine collection device, comprising:
a fluid impermeable barrier at least partially defining an opening and a chamber within the fluid collection device in fluid communication with the opening, the fluid impermeable barrier having a bottom portion at least partially defining the opening, a top portion opposite to the bottom portion, a proximal end region, and a distal end region, the opening being sized to receive at least a portion of a penis therethrough and positioned between the distal end region and the proximal end region;
a fluid permeable body positioned at least partially within the chamber between the top portion and the bottom portion of the fluid impermeable barrier and extending at least partially between the distal end region and the proximal end region; and
a tube extending into the chamber and having an end positioned proximate to the distal end region of the chamber, wherein the tube includes a tube opening at least proximate to the end of the tube and oriented to face at least a portion of the fluid permeable body with the tube opening spaced from the distal end region of the fluid impermeable barrier between the distal end region and the opening of the fluid impermeable barrier.
